(19) United States
(12) Patent Application Publication
(10) Pub. No.: US 2022/0110888 A1
(43) Pub. Date: Apr. 14, 2022

(54) COMPOSITION FOR THE TREATMENT OF PSORIASIS

(71) Applicant: Charlotte's Web, Inc., Boulder, CO (US)

(72) Inventors: Jeffrey Lokken, Buffalo, NY (US); Katherine Cwiklinski, Buffalo, NY (US); Sherry Bradford, Grand Island, NY (US)

(73) Assignee: Charlotte's Web, Inc., Boulder, CO (US)

(21) Appl. No.: 17/498,879

(22) Filed: Oct. 12, 2021

Related U.S. Application Data
(60) Provisional application No. 63/089,586, filed on Oct. 9, 2020.

(51) Int. Cl. A61K 31/05; A61K 45/06; A61K 47/44; A61P 17/06

(52) U.S. Cl. CPC A61K 31/05; A61P 17/06; A61K 47/44; A61K 45/06

(57) ABSTRACT

A composition for the treatment of psoriasis, wherein the composition comprises a CB2 receptor binding agent, an immunosuppressive agent an oil; and a healing agent or an anti-inflammatory agent. The CB2 receptor binding agent may be a hemp-derived cannabinoid isolate, a hemp-derived full-spectrum cannabinoid containing extract; a hemp-derived broad-spectrum cannabinoid containing extract, or mixtures thereof

### CROSS-REFERENCE TO RELATED APPLICATIONS

This application is a Nonprovisional application of pending U.S. Provisional Application No. 63/089,586, filed Oct. 9, 2020, the disclosure of which is incorporated herein by reference.

### FIELD OF INVENTION

This invention is directed to a composition for treating psoriasis. More particularly, the composition comprises at least one cannabinoid.

### BACKGROUND OF INVENTION

The skin is the body's largest organ and, as such, is home to a vast number and variety of diseases. One such disease is psoriasis, which is an immune-mediated, chronic inflammatory skin malady that causes scaly patches most commonly on the elbows, knees, trunk, and scalp. These patches, which become dry, red, and itchy, cause pain and discomfort to the suffering individual. Psoriasis can also result in psychiatric symptoms like reduced self-esteem and anxiety because it is a skin condition that is often visible on exposed parts of the body.

Psoriasis is common, affecting about 2% of the worldwide population. Chronic plaque psoriasis, referred to as psoriasis vulgaris is the most common type of psoriasis, manifested as dry, red raised plaques with adherent silvery scales. Psoriasis is characterized by hyper-proliferation and aberrant differentiation of keratinocytes, dilated, hyper-plastic blood vessels as well as an inflammatory infiltration of leukocytes, into the dermis.

Psoriasis is generally considered a genetic disease, thought to be triggered and influenced by environmental factors. Substantial clinical and basic research observations indicate that the cellular innate and adaptive immune responses, especially the activation of T cells, play a critical role in the pathogenesis of psoriasis.

Psoriasis causes a list of clinical phenotypes on the skin of its sufferers. It has been estimated to affect approximately five million people in the United States (NIH, 2009). Some of these symptoms include dermal patches that are red with silvery scales, dry, cracked skin that may bleed, itch, burn or be sore, swollen and stiff joints, and in children, small scaling spots. These patches often appear on the elbows, knees, lower back and scalp. Plaque psoriasis is the most common form of psoriasis but there are many types including; nail, guttate, inverse, pustular, erythrodermic, and psoriatic arthritis.

The causes of psoriasis are not fully understood. It is not purely a skin disorder and it can have negative impact on many organ systems. Psoriasis is also associated with an increased risk of certain cancers, cardiovascular disease, and other immune-mediated disorders such as Crohn's disease and ulcerative colitis.

While there is currently no known cure for psoriasis, there are prescription and over the counter treatments available to lessen symptom severity. Psoriasis treatments aim to stop skin cells from growing so quickly and to remove dry scales. Treatment options include creams and ointments (topical therapy), light therapy (phototherapy), and oral or injected medication. Common treatments are corticosteroids, retinoids, coal tar, cyclosporine, exposure to sunlight and biologics.

Conventional prescription treatments and conventional over the counter treatments have several disadvantages. Currently available conventional prescription drugs can take between 6-16 weeks to produce any noticeable changes. Topical treatments containing active ingredients such as coal tar and salicylic acid do show promise for certain individuals, but the overall efficacy is highly variable. In addition, many conventional medical treatments have negative side-effects such as fatigue, loss of appetite, upset stomach, potential for kidney damage, and high blood pressure.

When treating psoriasis, the goal is to find the most effective way to slow cell turnover with the fewest possible side effects. All of the above-listed treatments have side effects ranging from thinning of the skin, skin irritation, increased sensitivity to light, to suppression of the immune system, and increased risk of serious infection.

Moreover, the above-listed treatments aren't thoroughly effective. Because the triggers for psoriasis onset are numerous and often unavoidable, it is virtually impossible to halt the induction of the symptoms before they appear on the skin. For example, in some cases, psoriasis can be caused by an autosomal dominant genetic trait that is associated with allele HLA-Cw6. (Alshobaili H A, Shahzad M, Al-Marshood A, Khalil A, Settin A, Barrimah I. Genetic background of psoriasis. Int J Health Sci (Qassim). 2010; 4(1):23-29). In other cases, environmental triggers of psoriasis include; infections, diet, consumption of dairy or alcohol, skin injuries, stress, cold weather, smoking, and medications. The innate and adaptive immune systems respond to these triggers with an activation of an immune response which cause cytokine storms and results in the continuous cycle of psoriasis outbreaks.

Importantly, many individuals are not happy with the treatment of their psoriasis. Individuals may suffer from extensive side effects or may have incomplete treatment of the psoriasis.

Thus, there is a need for psoriasis treatment that minimizes side effects while improving the overall treatment results.

### SUMMARY OF THE INVENTION

Accordingly, it is the subject of this invention to provide a composition for the treatment of psoriasis that addresses all of the biological pathways and mechanisms involved in the initiation, activation, and manifestation of psoriasis. The composition of the present disclosure is a holistic and balanced approach to treating psoriasis.

### BRIEF DESCRIPTION OF THE DRAWINGS

FIG. 1 depicts a table comparing IL-23 expression results.

FIG. 2 depicts a table comparing IFN-γ expression results.

FIG. 3 depicts a table comparing CD-28 expression results.

FIG. 4 depicts a table comparing IL-6 expression results.

FIG. 5 depicts a table comparing IL-23 expression results.

FIG. 6 depicts a table comparing IL-17a expression results.

FIG. 7 depicts a table comparing IFN-gamma expression results.

FIG. 8 depicts a table comparing TNF-alpha expression results.

FIG. 9 depicts a table comparing MMP-9 expression results.

FIG. 10 depicts a table comparing Substance P expression results.

FIG. 11 depicts a table comparing VEGF expression results.

FIG. 12 depicts a table comparing Toll-Like Receptor-2 expression results.

FIG. 13 depicts a table comparing IL-6 expression results.

FIG. 14 depicts a table comparing Caspase expression results.

FIG. 15 depicts a psoriasis area and severity index table.

FIG. 16 depicts a dermatology life quality index table.

FIG. 17 depicts a universal pain assessment tool.

FIG. 18 depicts a pain index table.

FIG. 19 depicts a pruritus table.

FIG. 20 depicts a caliper table.

FIG. 21 depicts an erythema via hexameter table.

FIG. 22 depicts a hydration via corneometer table.

FIG. 23 depicts a visioscan table.

FIG. 24 depicts visioscan images for patient CW 1001.

FIG. 25 depicts visioscan images for patient CW 1002

FIG. 26 depicts patient photographs for patient #CW 1001.

FIG. 27a depicts patient photographs for patient #CW 1002.

FIG. 27b depicts patient photographs for patient #CW 1002.

### DETAILED DESCRIPTION OF THE INVENTION

In one embodiment, the composition is comprised of: at least one cannabinoid selected from the group consisting of cannabidiol (CBD), cannabigerol (CBG), tetra hydrocannabioid (THC), cannabinol (CBN); alpha bisabolol; ethyl linoleate; cholecalciferol; ascorbic acid; at least one terpene or terpenoid; at least one ceramide; oil; allantoin; palmitoylethanolamine (PEA); and oleoylethanolamide (OEA).

It is noted that cholecalciferol is also referred to as vitamin D3 and ascorbic acid is also referred to as vitamin c. Ascorbic acid includes stable derivatives such as ascorbyl palmitate. Terpenes include terpenoids and terpene alcohols. Exemplary oils include coconut oil and hemp oil.

In one embodiment, the cannabinoids may exist as individual isolates or distillates or in full spectrum hemp/cannabis extract form or any combination thereof. In an alternative embodiment, hemp may be used instead of the cannabinoids.

In one embodiment, the terpenes used may be added separately to the composition or may exist in full spectrum hemp, or broad spectrum CBD or may be in the form of terpenes derived from other plants.

In a preferred embodiment, the oil is coconut oil and functions as a carrier oil for the composition and also has the capability to reduce red raised plaques and relieve dry skin. It is understood that other cosmetic and topical oils may serve a similar function.

Ethyl linoleate is a more shelf stable version of linoleic acid, but, in alternative embodiments, linoleic acid may be used.

In some embodiments, not all of the above-listed ingredients may be used in the composition.

In another preferred embodiment, the composition is delivered topically by way of an anhydrous carrier base. This embodiment will be referred to as Psoriasis Spray or (PS). In another embodiment, the composition is delivered as a water-based emulsion. There are two versions of this embodiment referenced in the in-vitro data section, Psoriasis Cream (PC) and Psoriasis Cream OTC (PCOTC). It is understood that any suitable carrier base may be used to deliver the topical. In another embodiment, the topical may be used along with an ingestible comprising the same or similar composition.

Possible Mechanism of Action

Without intending to be bound by theory, the composition works by halting dendritic dermal cells from proliferating and producing a cytokine release syndrome (also known as cytokine storm) by reducing the potential for proliferation. This causes a reduction of the cytokines IL-23 and TNFα which would eventually lead to an autoimmune response. Ingredients in the composition that target the innate pathway proliferation of dermal dendritic cells are: cannabinoids, CBD, CBN, THC, and terpenes.

It is believed that there are certain compounds capable of suppressing the activation of pathogenic Tc cells. When activated, Tc cells differentiate into intraepidermal CD8+ and CD4+ T-cells which produce pathogenic cytokines such as IL-23. The outcome of Tc activation being left untreated causes memory T cells to be produced which repeatedly generate an accelerated and robust antibody-mediated immune response. The goal here is to stop this part of the pathway, thereby reducing and possibly preventing the manifestations of the disease.

In-Vitro Data

In-vitro experiments using the previously described compositions were performed using human jurkat T-cells, human primary keratinocytes and human primary schwann cells to characterize and quantify the cellular changes in gene expression that occurred after 24-hour treatment of the present composition. All experiments were performed in triplicate on 24-well culture plates and treated for 24-hours before RNA was isolated using Trizol. Reverse transcriptase polymerase chain reaction (rtPCR) was performed on all samples and subsequentially, all samples were analyzed using quantitative polymerase chain reaction (qPCR).

Although the pathogenesis of psoriasis is not fully understood, there is ample evidence suggesting that the dysregulation of immune cells in the skin, particularly T cells, plays a critical role in psoriasis development. Psoriasis is considered to be an organ-specific T cell-driven inflammatory disease and T cells play a dominant pathogenic role in the initiation and maintenance of psoriasis. (Cai Y, Fleming C, Yan J. New insights of T cells in the pathogenesis of psoriasis. Cell Mol Immunol. 2012; 9(4):302-309). Human jurkat T-cells were used in an in-vitro experiment comparing three versions of the composition, Psoriasis Spray (PS), Psoriasis Cream (PC) and Psoriasis Cream OTC (PCOTC) as well as an over-the-counter competitive national brand psoriasis treatment containing 3% salicylic acid and referred to as (AP).

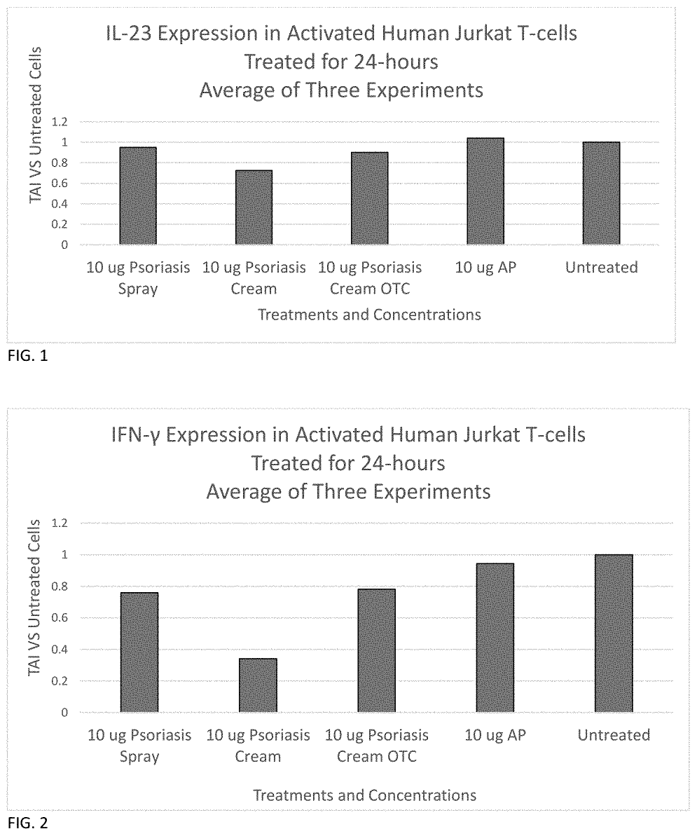

The inflammatory signaling interleukin referred to as IL-23 acts as modulator of T helper cells, directing these helper immune cells to release further signaling cytokines which allow the host to develop a more precise response plan depending on the antigen. In addition, IL-23 signaling induces CD4 memory T cell propagation and in doing so, encourages a chronic state of autoinflammation in a positive feedback cycle. Perhaps more consequentially, IL-23 is directly correlated to occurrences of angiogenesis, a main agitator in the development of psoriasis. As shown in FIG. 1, downregulation of this inflammatory cytokine was achieved after treatment with 10 ug Psoriasis Spray (PS) and for both topical Psoriasis Cream (PC) and Psoriasis Cream OTC (PCOTC) by 5%, 10% and 27% respectively compared to the untreated control. All three formulations out preformed the competitive product (AP) which increased production of IL-23 by 4%.

The cytokine interferon gamma (IFN-γ) plays a central role in the pathology and mechanism of psoriasis as circulating levels show a direct, positive correlation to between legion size and severity index according to a recent study from Medical Archives. (Kurtovic N O, Halilovic E K. Serum Concentrations of Interferon Gamma (IFN-γ) in Patients with Psoriasis: Correlation with Clinical Type and Severity of the Disease. Med Arch. 2018; 72(6):410-413). 10 ug of Psoriasis Cream demonstrated the most effective reduction in production of IFN-γ, followed by 10 ug Psoriasis Spray and Psoriasis Cream OTC ranging from 22% to 66% compared to untreated controls while AP showed just a 6% knockdown in activated human jurkat T-cells. This is shown in FIG. 2.

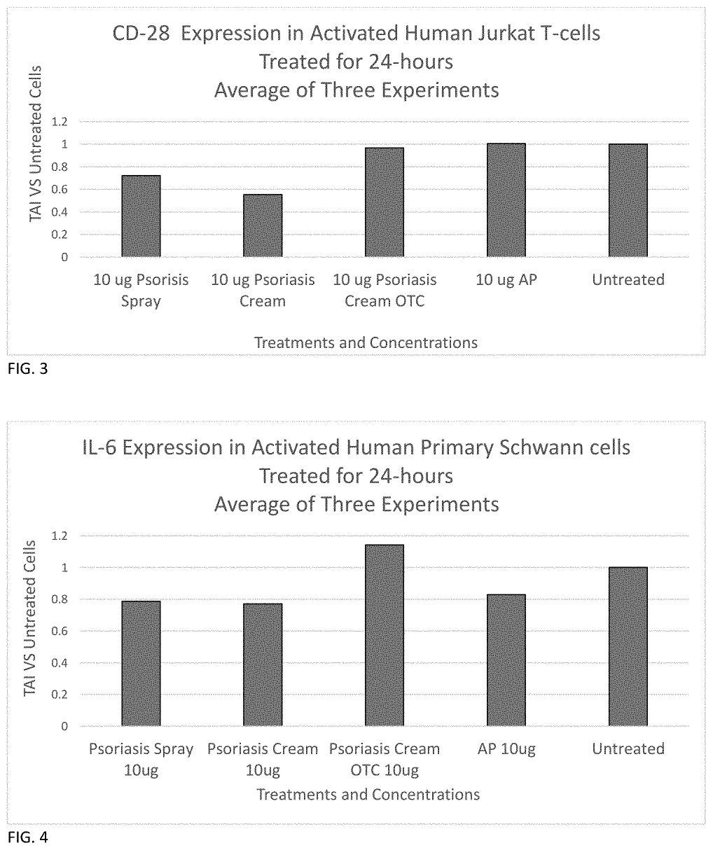

Cluster of Differentiation 28 (CD-28) is a protein expressed on the surface of T cells which mediates signals responsible for T cell activation while in the proximity of antigen-presenting cells. Stimulation of T cells by this receptor induce production of inflammatory cytokines and interleukins which give rise to immune-mediated inflammatory diseases such as psoriasis according to the British Journal of Dermatology. (Lima, X., Cintra, M., Piaza, A., Mamoni, R., Oliveira, R., Magalhães, R. and Blotta, M. (2015), Frequency and characteristics of circulating CD4+ CD28null T cells in patients with psoriasis. Br J Dermatol, 173: 998-1005). As such, diminished expression here should mitigate activation and the T cell mediated response which ultimately elicit systemic inflammation and by extension, psoriatic legions of the epidermis. FIG. 3 shows the knockdown of CD-28 was reportedly greatest after treatment with 10 ug of the Psoriasis Cream at 45%, followed by 10 ug of the Psoriasis Spray and the Psoriasis Cream OTC at 28% and 3% respectively while the competitor product does not affect CD-28 expression compared to the control group of Untreated cells.

Schwann cells, the cells responsible for producing the protective layer of insulation known as myelin around axonal nerve fibers are of the first cells to launch an initial response to nerve damage via regeneration. After nerve injury occurs, Schwann cells will lose contact with their accompanying axon, thus altering their signaling environment in such a way to trigger the release of inflammatory molecules which in turn activate nociceptive neurons directly responsible for neuropathic pain. Of these secreted inflammatory signaling molecules is IL-6, a significantly upregulated biomarker in psoriatic lesions compared to healthy controls. Upregulation here is suspected to hinder the function of regulatory T cells in suppressing autoinflammation. Perhaps of greater consequence, it has been recently hypothesized that continual activation of Schwann cell repair processes as in the process previously described further promotes the positive feedback loop responsible for the autoimmune nature of psoriasis. (Goodman W A, Levine A D, Massari J V, Sugiyama H, McCormick T S, Cooper K D. IL-6 signaling in psoriasis prevents immune suppression by regulatory T cells. J Immunol. 2009; 183(5):3170-3176. doi:10.4049/jimmunol.0803721). We tested our present composition on activated human primary schwann cells to quantify the changes in gene expression known to contribute to psoriasis. FIG. 4 shows treatment with 10 ug of the present composition versions, Psoriasis Spray and Psoriasis Cream effectively knocked down IL-6 expression in these activated cells by up to 23% and 22% respectively and out preforming our top competitor (AP) tested at the same concentration.

Another inflammatory signaling molecule secreted by Schwann cells that contributes to psoriatic pathogenesis is IL-23. This interleukin activates T helper cells to initiate their own signaling cascade via IL-17A, IL-17F, and IL-22. Exposure of these proinflammatory mediators on keratinocytes of the epidermis directly induces hyperproliferation and by extension, furthering the severity of outbreaks. Treatment with 10 ug of Psoriasis Cream averaged a 17% knockdown in IL-23 expression compared to untreated controls, far out preforming the competitor product (AP) against the same testing parameters which increased IL-23 expression by 23%. This data shown in FIG. 5, viewed together with data showing a knockdown of IL-23 in human jurkat T-cells is un-expected result and may explain why the present composition is effective in reducing psoriasis.

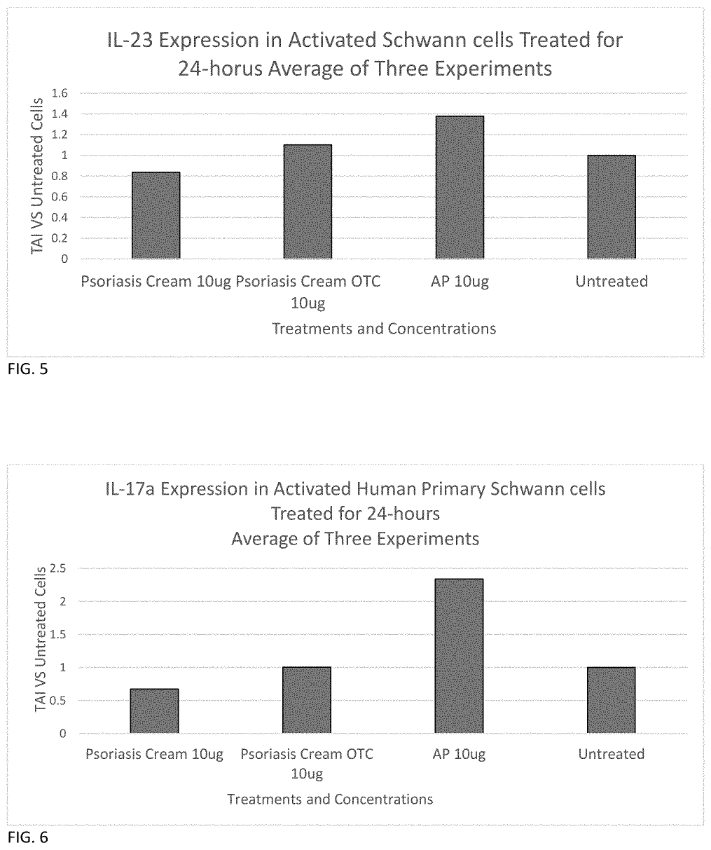

Interleukin 17A (IL-17A) is one of the currently known six members of the IL-17 cytokine family and is implicated in immune responses to infectious pathogens and in the pathogenesis of inflammatory autoimmune diseases like psoriasis. IL-17A together with other Th17 cytokines also upregulates the production of several chemokines that are implicated in psoriasis pathogenesis. IL17A-targeting antibodies show an impressive clinical efficacy in patients with psoriasis. (von Stebut Esther, Boehncke Wolf-Henning, Ghoreschi Kamran, Gori Tommaso, Kaya Ziya, Thaci Diamant, Schaffler Andreas. IL-17A in Psoriasis and Beyond: Cardiovascular and Metabolic Implications. Frontiers in Immunology. Vol: 10, 2020.) Reducing the expression of the protein IL-17A would in turn decrease Schwann cell exposure to the molecule and in doing so, decrease recovery times of psoriasis improvement. 10 ug of Psoriasis cream effectively knocked down IL-17a by an average of 33% compared to untreated controls, even more dramatically, an increase in production of this protein was found after treatment of the top competitor (AP), which increased IL-17a production by 133%, as shown in FIG. 6.

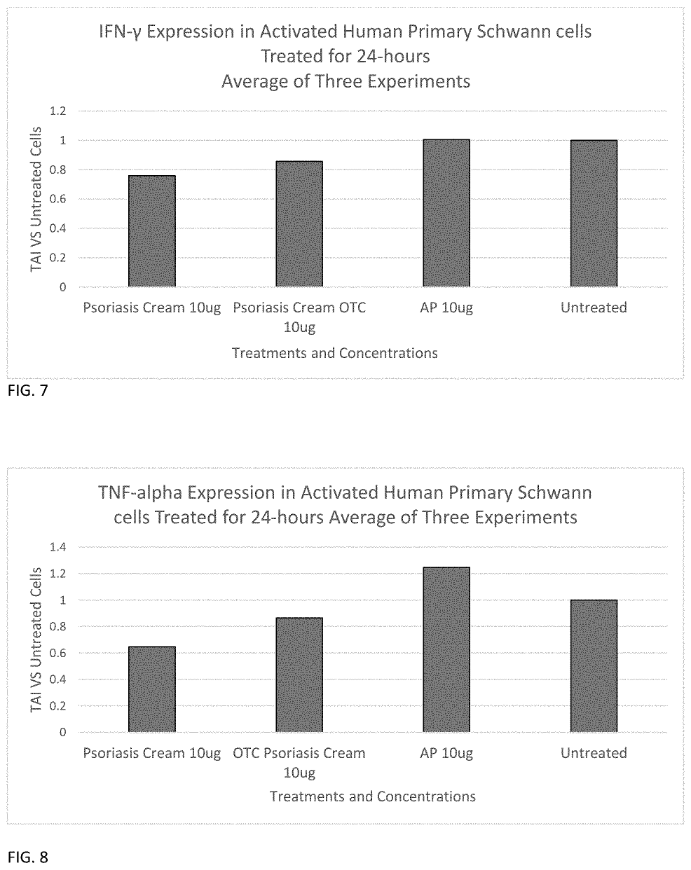

Interferon gamma or IFN-γ is yet another inflammatory signaling molecule secreted by Schwann cells that is primarily known for its function in activating macrophages in the presence of a pathogen. Aside from detecting, engrossing, and destroying pathogenic material, macrophages also initiate further signal cascades to activate neighboring cells to the threat and in some cases, present the pathogen to them. (Choi, K. et al. An inflammatory gene signature distinguishes neurofibroma Schwann cells and macrophages from cells in the normal peripheral nervous system. Sci. Rep. 7, 43315; doi: 10.1038/srep43315 (2017)). Limiting macrophage activation by reducing the circulation of IFN-γ in a system is thus imperative to preventing an autoimmune attack such as in the case of psoriatic lesions. FIG. 7 shows treatment with 10 ug of both the psoriasis cream and OTC cream demonstrated knockdown efficacy of roughly 10-25% for expression of this protein in Schwann cells compared to untreated controls where the competitor product (AP) did not affect IFN-γ.

TNF-alpha, or tumor necrosis factor-alpha, is a dynamic cytokine involved in the development and continuation of psoriasis as well as a potent activator of schwann cells and other cells such as T-cells. Selectively blocking the role of this cytokine has proven highly effective at mitigating psoriasis in all of its manifestations in both the skin and joints. (Tobin A M, Kirby B. TNF alpha inhibitors in the treatment of psoriasis and psoriatic arthritis. BioDrugs. 2005; 19(1):47-57). On treating activated human primary schwann cells with 10 ug of both the Psoriasis Cream and Psoriasis Cream OTC, knockdown of TNF-alpha was found to be 36% to 14% respectively compared to controls and with great efficacy, unlike that of the top competitor, which showed an increase in expression on TNF-alpha by 24%. This result can be found in FIG. 8.

Matrix metalloprotein-9 (MMP-9) is an enzyme predominantly involved in the degradation of the extracellular matrix, forming new blood vessels and promoting leucocyte migration into the dermal layer. Due to the nature of MMP-9 as an inflammatory cell activator, there is a positive correlation between upregulation of this enzyme and the intensity of inflammation in the areas of psoriatic outbreak. (Amezcua-Guerra L M, Bojalil R, Espinoza-Hernandez J, Vega-Memije M E, Lacy-Niebla R M, Ortega-Springall F, Ortega-Hernández J, Sánchez-Munoz F, Springall R. Serum of Patients with Psoriasis Modulates the Production of MMP-9 and TIMP-1 in Cells of Monocytic Lineage. Immunol Invest. 2018 October; 47(7):725-734). Both the Psoriasis Cream and Psoriasis Cream OTC far outperformed the top competitor (AP) under the same conditions, knocking down expression by 17% and 7% respectively when comparing to untreated controls while the competitor product increased expression of MMP-9 by 75%. This data is found in FIG. 9.

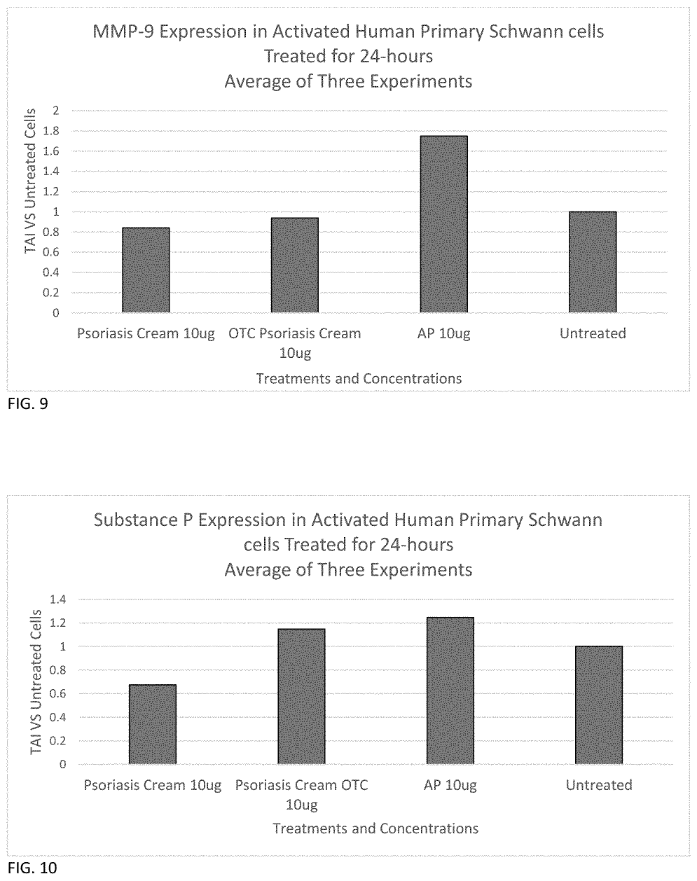

Substance P is neuropeptide expressed by cells of the peripheral nervous system responsible for initiating cutaneous inflammation by activating immune cells such as T cells and macrophages, resulting in itch and the sensation of pain. Downregulation here not only inhibits hyperproliferation of keratinocytes directly related to psoriatic lesions but also curbs dermal inflammatory response. (Tom C. Chan, Meng-Sui Lee, Wen-Chih Huang, Wen-Yu Chang, James G. Krueger, Tsen-Fang Tsai, Capsaicin attenuates imiquimod-induced epidermal hyperplasia and cutaneous inflammation in a murine model of psoriasis, Biomedicine & Pharmacotherapy, 10.1016/j.biopha.2021. Ser. No. 11/950,141, (111950), (2021)). FIG. 10 shows the result of treatment with 10 ug of Psoriasis Cream, which knocked down substance P expression by 33% on average compared to untreated controls and, dramatically, compared to the competitor product (AP) which showed an increase in substance P production by 24%.

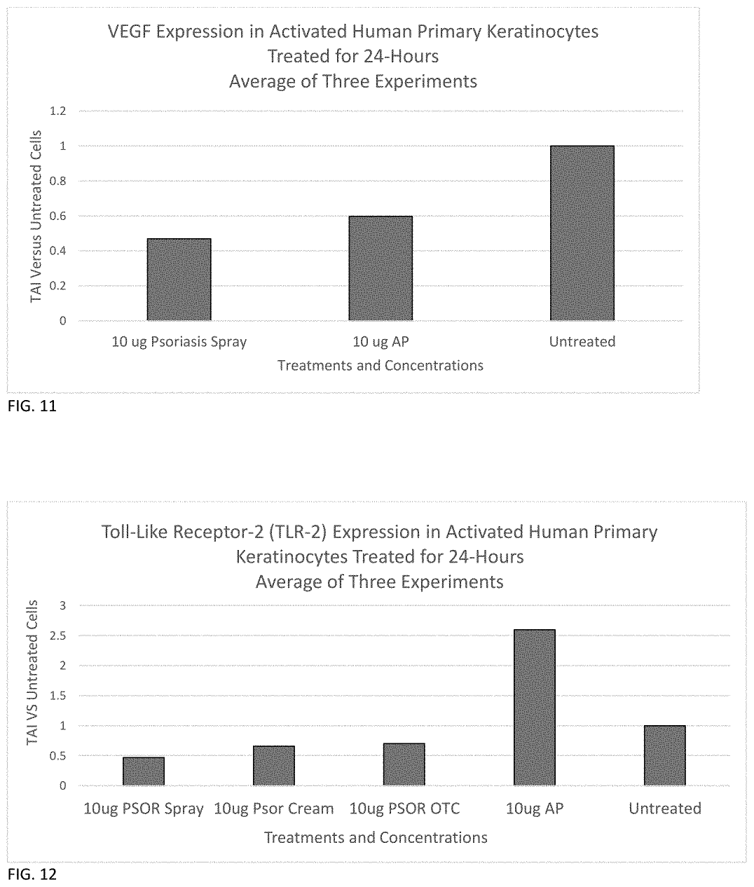

Proangiogenic VEGF is the main factor overexpressed in psoriatic skin. VEGF can be released by activated keratinocytes and immune cells (Canavese M, Altruda F, Ruzicka T, Schauber J. Vascular endothelial growth factor (VEGF) in the pathogenesis of psoriasis-a possible target for novel therapies? J Dermatol Sci. 2010 June; 58 (3):171-6.), contributing to an increase in the density of psoriatic dermal capillaries, in addition to enhanced permeability and dilatation. In FIG. 11, we show that a 10 ug treatment of Psoriasis Spray reduces VEGF expression by 50%, while the competitor product did also reduce the expression by just 40%.

Toll-like receptor 2, or TLR-2, functions as a pattern recognition receptor for a wide variety of antigens that initiate the innate immune system signaling cascade. Activation of TLR-2 on the surface of keratinocytes directly correlates with an increase of apoptotic staged cells and as a result, serves as a means for manifesting nerve damage. (Jamie E. McInturff, Robert L. Modlin, Jenny Kim, The Role of Toll-like Receptors in the Pathogenesis and Treatment of Dermatological Disease, Journal of Investigative Dermatology, Volume 125, Issue 1, 2005, Pages 1-8.) Considering this, it is no surprise that TLR-2 demonstrates differential expression at sites of various dermatological diseases including psoriasis, acne and leprosy to name a few. In a triplicate experiment, a 10 ug treatment of the Psoriasis Spray, Psoriasis Cream and Psoriasis Cream OTC had significantly diminished expression of the inflammatory biomarker by 53%, 34% and 30% respectively, especially when compared to the top competitor (AP) which showed a 163% increase in the expression of TLR-2. This data can be seen in FIG. 12.

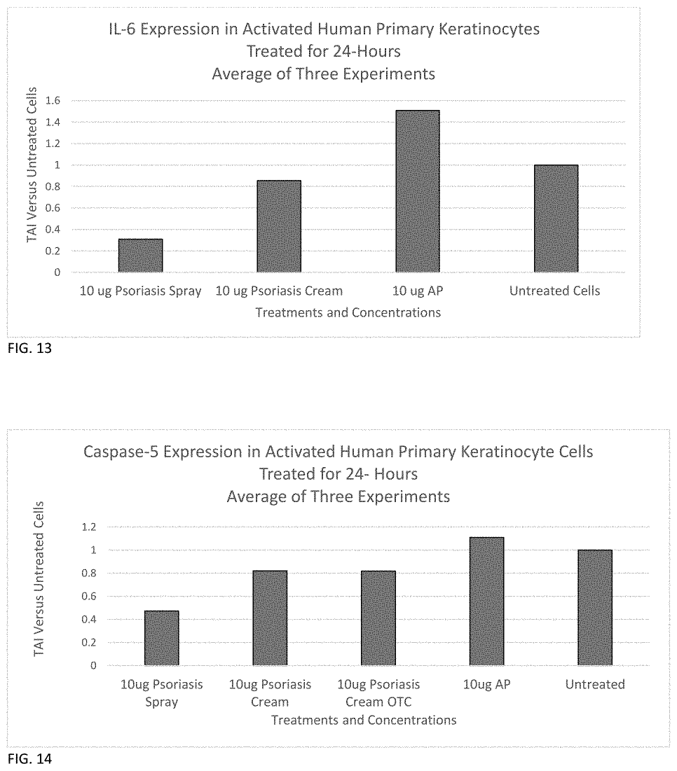

FIG. 13 shows treatment with 10 ug of the present composition versions, Psoriasis Spray and Psoriasis Cream effectively knocked down IL-6 expression in activated human primary keratinocytes by up to 80% and 43% respectively and out preforming our top competitor (AP) tested at the same concentration.

In a study conducted by the Journal of Investigative Dermatology, in vitro experiments revealed that caspase-5 mRNA was induced in primary keratinocytes as well as PBMCs stimulated with IFN-g. Inhibition studies suggested that caspase-5 mRNA upregulation was mediated through the NF-kB pathway. This suggests that caspase-5 and the inflammasome may have an important role in the inflammatory response in psoriasis. (Caspase-5 Expression Is Upregulated in Lesional Psoriatic Skin, Journal of Investigative Dermatology (2011) 131, 670-676.) Caspase-5 expression was reduced after treatment with 10 ug of Psoriasis Spray, Psoriasis Cream and Psoriasis Cream OTC with a reduction in expression of 52%, 18%, and 18% respectively. The competitor product showed an increase of expression by 10%. This is shown in FIG. 14.

The composition of the present disclosure is effective in treating pain, dry skin, psoriatic arthritis, bleeding, red raised plaques, silvery scales, dilated hyperplastic blood vessels, angiogenesis, scarring, puritis, and general inflammation.

Benefits of the Composition

The composition of the present disclosure is a holistic and natural broad-acting hemp unguent. It is believed that, by supplying a continuum of healing molecules, the composition is anti-inflammatory. It is also believed to prevent the scarring that is generally associated with the psoriasis healing process.

Preclinical Evaluation of Efficacy

To assess the efficacy of the composition of this disclosure, investigators recruited two female participants that had previously been diagnosed with chronic psoriasis. In both participants, the chronic psoriasis was currently active. Active psoriasis is defined by lesions and/or scales that are causing pain, itching, and/or discomfort. At the time of the investigation, neither participant was using any other product or medication for psoriasis relief.

Both participants were provided with a CBD-containing lotion for topical use, as well as a CBD tincture for systemic use. By applying the lotion to the skin, CBD is theorized to stimulate the subcutaneous endocannabinoid system. Because CBD does not reach the blood stream from topical use, a CBD tincture was used as a means of reaching the systemic endocannabinoid system in tandem with the subcutaneous endocannabinoid system.

Psoriasis Cream is a hydrating topical formulation that contains hemp extract. In one embodiment, full spectrum hemp extract is used. This extract contains a variety of phytocannabinoids, terpenes, and flavonoids. In a preferred embodiment, the level of full spectrum hemp extract in the lotion is 400 mg per ounce, with approximately 200 mg of that being CBD. The psoriasis relief lotion also includes CBD isolate. The CBD isolate is added at 200 mg per ounce, bringing the total CBD quantity to 400 mg per ounce. This is considered to be a high level of CBD for a topical product. However, we have shown that CBD does not reach the bloodstream when it is introduced into the system by way of dermal application. CBD will penetrate the skin and reach multiple layers, but it will never cross over into systemic circulation. Thus, this high-level product is safe for daily use. In this investigation, the amount of CBD applied per day was variable depending on the number of affected areas being treated and the volume of lotion used to cover each area.

In one embodiment, the CBD tincture is also made with full spectrum hemp extract and contains 60 mg hemp extract per 1 milliliter. The participants were advised to take one dropper (0.5 mL) three times per day. This made their internal dosage of hemp extract 90 mg per day, with approximately 45 mg of that being CBD.

Investigators surveyed the participants' progress by assessing a variety of parameters weekly, for a total of twelve weeks. Week 0 refers to the baseline, or initial, readings and scores that were obtained for the participant prior to any treatment. For clarity, we have shown the resulting data obtained at the end of each 4-week segment of treatment.

In this study investigators utilized reliable dermatological assessments of pain, pruritis, and quality of life. These are well studied standards for assessing the subjective experience of the disorder. Since the severity of these symptoms is not testable by conventional methods, investigators must rely on the patient's recounting of the symptoms. The investigators also assessed erythema, or redness due to inflammation, hydration, and topography of the skin at the affected areas using sophisticated skin analytical equipment.

Psoriasis Area and Severity Index

The Psoriasis Area and Severity Index (PASI) is the single most used tool for the measurement of psoriasis severity. Clinicians use PASI to generate a score of psoriasis severity, on a scale of 0-72, by surveying the percent of skin area involved, erythema (redness), induration (thickness), and desquamation (scaling). Although an initial PASI score is often used to determine a patient's entry into a trial, it is response to treatment that is important to measure efficacy and outcomes. It is currently considered the gold standard for assessing treatment efficacy in moderate-to-severe psoriatic patients.

Psoriasis Area and Severity Index

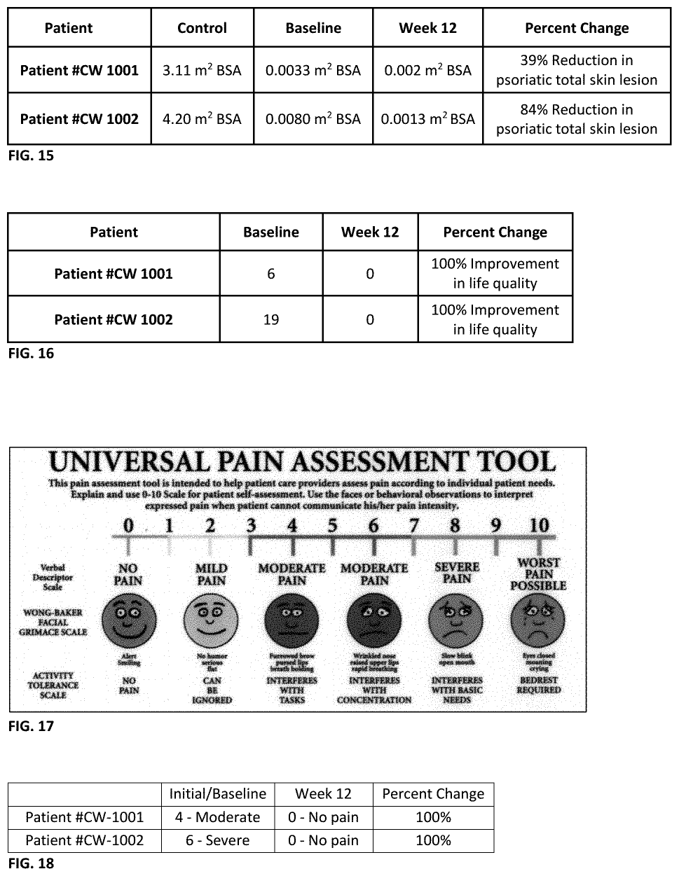

Body surface area (“BSA”) is an estimate of the surface area of the individual body's skin. This is a relative calculation of the volume of skin in total. When BSA is compared with the PASI it indicates the psoriatic surface area treatment efficacy. The National Psoriasis Foundation identified the acceptable response for plaque psoriasis after three months of treatment as either less than 3 percent body surface area (BSA) involvement or 75 percent improvement compared with baseline. As shown in FIG. 15, CW 1001 experienced less than 3% BSA involvement. CW 1002 experienced greater than 75% improvement in psoriasis total body lesion.

Dermatology Life Quality Index The Dermatology Life Quality Index (“DLQI”) is a widely used tool for assessing a patient's subjective quality of life when suffering from a dermatological condition. This is a ten-question survey in which a patient is asked to rate how much or how little their condition interferes with their quality of life. The questions survey how the condition effects daily activities, personal relationships, and mental health. The four answer choices are rated on a scale of zero to three; zero being no effect or irrelevant, one being a little effect, two being a lot, and three being very much effected. This allows for a maximum resulting score of 30 which may also be expressed as a percentage. The higher the score the more quality of life is impaired. A score higher than 10 indicates that the patient's life is being severely affected by their disease. For general inflammatory skin conditions, a change in DLQI score of at least four points is considered clinically important. As shown in FIG. 16, both patients experienced a 100% improvement in life quality after twelve weeks of treatment.

Pain

Pain is an unpleasant sensory and emotional experience associated with actual or potential tissue damage. Pain is always subjective and can only be recorded by the individual's reported experience. Medical providers utilize the subjective Universal Pain Assessment Tool (“UPAT”), as shown in FIG. 17, to assess pain and tend to patient needs. In this investigation, both participants were asked to rate their relative pain index each week as shown in FIG. 18. By the end of the 12-week period, both participants responded with a zero rating for pain. Accordingly, it appears that there was a 100% recovery in pain experience for both CW 1001 and CW 1002.

Pruritis

Pruritis, or itching, is another one of the most common symptoms of psoriasis. The immune system becomes overactive therefore epithelial (skin) cells will proliferate at a very fast rate. The sensory response stimulates immune system cells to become activated and secrete pro-inflammatory proteins, called cytokines. Full spectrum hemp extract contains a variety of phytocannabinoids, terpenes, and flavonoids. These molecules act on our nervous system by binding to brain sensory receptors. This activation attenuates the activation of skin sensory neurons, decreasing the itching response. The investigators hypothesized that the full spectrum hemp containing products would instigate such a reaction.

Pruritis, like pain, is a symptom that is completely subjective. To record a patient's relative level of itchiness, investigators employed the Visual Analog Scale (“VAS”). VAS is a tool used by clinicians in which the patient rates on a 0-10 scale their itch, zero being “no itch” and ten being “worst imaginable itch.” Determination follows the trend of: mild pruritus ≥0 but <3 points, moderate pruritus ≥3 but <7 points, severe pruritus ≥7 but <9 points, and very severe pruritus ≥9 points.

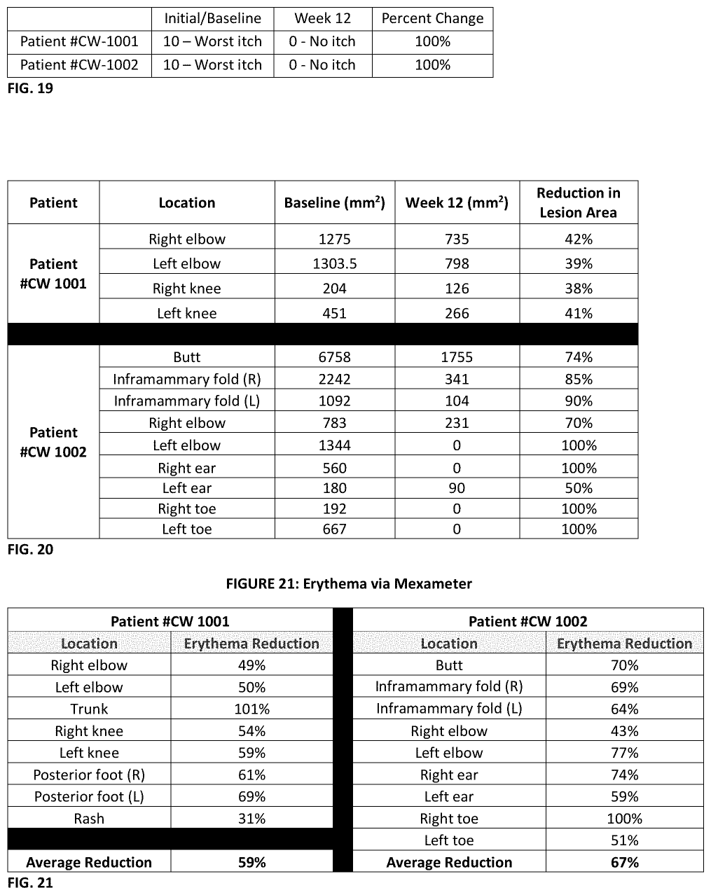

As shown in FIG. 19, both patients experienced a 100% percent reduction in pruritis. Given that both patients rated their baseline itchiness as a maximum ten, this is highly significant.

Caliper

The inflammation site can be measured using a caliper. If the area is relatively circular, the diameter at the greatest point is measured. If oblong, the widest point of length and width is measured. In this study, the inflammation site, or lesion, was oblong for all areas for both participants as depicted in FIG. 20. Thus, the length and width of the lesion to find the area of the affected location was measured.

Erythema Via Mexameter

Erythema refers to superficial reddening of the skin due to injury or irritation causing dilatation of the blood capillaries. Using a specialized piece of skin analytical equipment, investigators can measure this redness on an arbitrary but reputable scale. Courage & Khazaka electronics company has an advanced skin testing device known as the Cutometer® dual MPA 580. One of the respective skin testing probes which was used in this experiment is the Mexameter® MX 18. This probe measures erythema content by light absorption and reflection. Three wavelengths of light are emitted and the light reflected off the skin is measured by an internal receiver. The level of erythema is rated on a scale of 0-999.

It must be noted that accurate baseline readings of erythema were not recorded for every location during week zero. Both patients had lesion areas that were overcome with too severe of white scales. For these areas investigators were required to use their erythema measurement from week 1 of treatment, or in one case week 2, when the white flakes were off and therefore a true reading of redness was obtained. Another important note is that the heels of patient #CW 1001, which were monitored and treated during this study, were not included in the erythema survey. Due to an excessively thick layer of dense white scales, the Mexameter probe could not record an accurate reading of redness on this patient's heels.

For both participants, as shown in FIG. 21, there was a significant reduction in erythema at every tested location. Patient #CW 1001 experienced an average of 59% reduction in erythema and patient #CW 1002 an average of 67%.

Hydration Via Corneometer

The Corneometer® 825 probe has the unique ability to determine the surface hydration of the skin. It measures skin moisture based on the capacitance of skin due to its properties as a dielectric medium. The dielectric constant of the skin changes based on its current moisture level. The probe uses a measuring capacitor to record the smallest change in capacitance due to changes in the water content of the stratum corneum and displays it in arbitrary Corneometer units.

Improvement in hydration was determined by finding the relative change from baseline to final measurement.

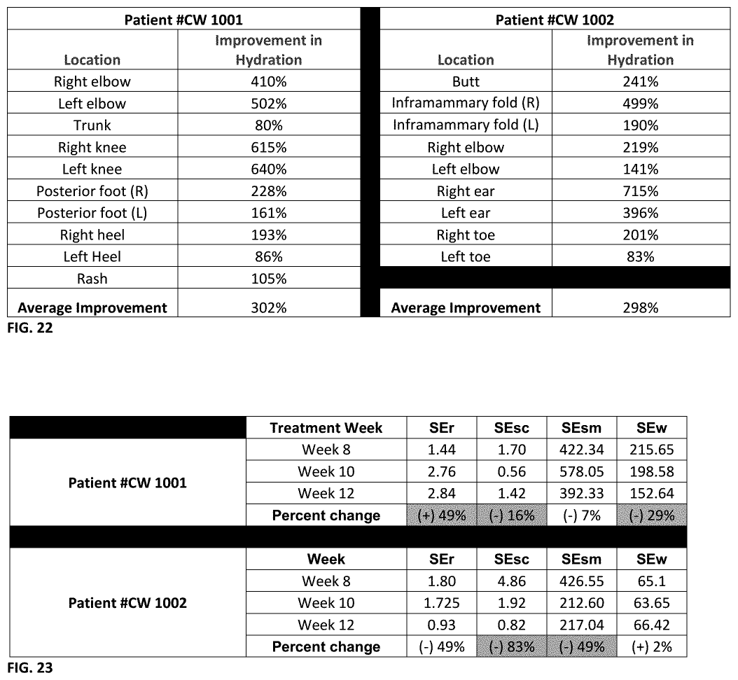

The improvement in skin hydration was clinically significant for both patients. The average improvements for patient #CW 1001 and #CW 1002 were 302% and 298%, respectively. This is depicted in FIG. 22.

Visioscan

The Visioscan VC20 Plus is a sophisticated instrument used to characterize skin condition in reference to four parameters: skin roughness (SEr), scaliness (SEsc), smoothness (SEsm), and wrinkles (SEw). It is equipped with a LED UV-A light video camera that allows for high resolution imaging and skin surface analysis. The software is capable of calculating the parameters using the grey level distribution from the photograph. Each pixel belongs to a specific grey level and these are quantified.

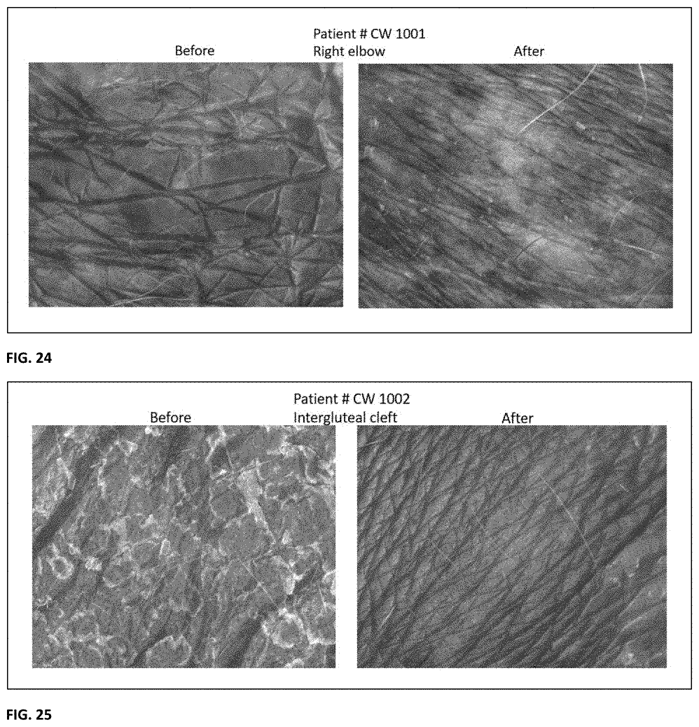

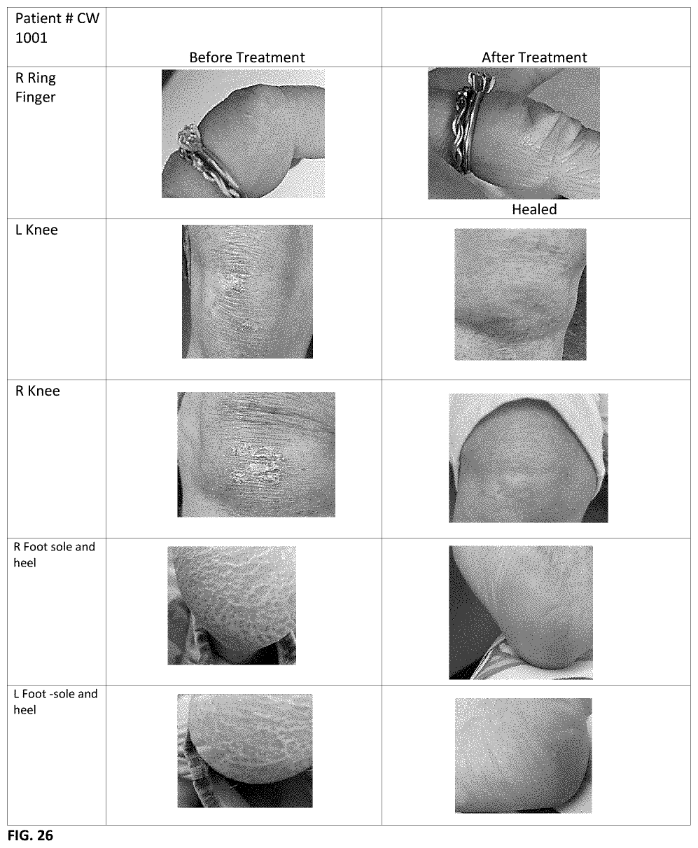

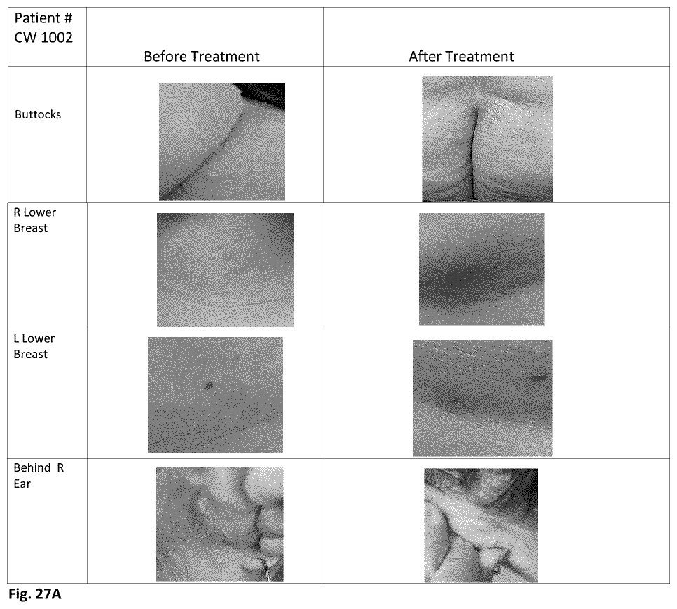

The Visioscan was probe was only utilized it for a small amount of supplemental data as shown in FIGS. 24 and 25. Investigators evaluated only the worst lesion for both participants. For patient #CW 1001, the right elbow was analyzed. For patient #CW 1002, the intergluteal cleft was analyzed.

The topography measurements are an average of three replicates per week. It should be noted that the Visioscan parameters are dependent on one another to a certain degree. In any one experiment, some parameters may improve while others do not change or appear to worsen. The key here is that the four set should be used as a unit and while some are improving, other measurements may be affected by compensation in the calculation. SEr is a measurement of skin roughness, it is hoped that a positive percent change occurs due to the inverse relationship between the expected parameter result and math. Scaliness (SEsc) and wrinkles (SEw) will have a negative percent change as expected when they improve. Smoothness (SEsm) also has an inverse relationship with its measurement, with negative percent change indicating improvement in skin smoothness.

Patient #CW 1001 had improvements in SEr, SEsc, and SEw. Most notable was the improvement in roughness at 49%. Scaliness and wrinkles also improved, while smoothness saw a very minor decline. Patient #CW 1002 had a profound result for scaliness, ending week 12 with 83% improvement. Their smoothness measurement was also significant, being overall improved by 49%. Wrinkles had no change, but roughness did appear to worsen. Overall, these results are positive as the improvements offset the one deficit. These results are depicted in FIG. 23.

Photographs

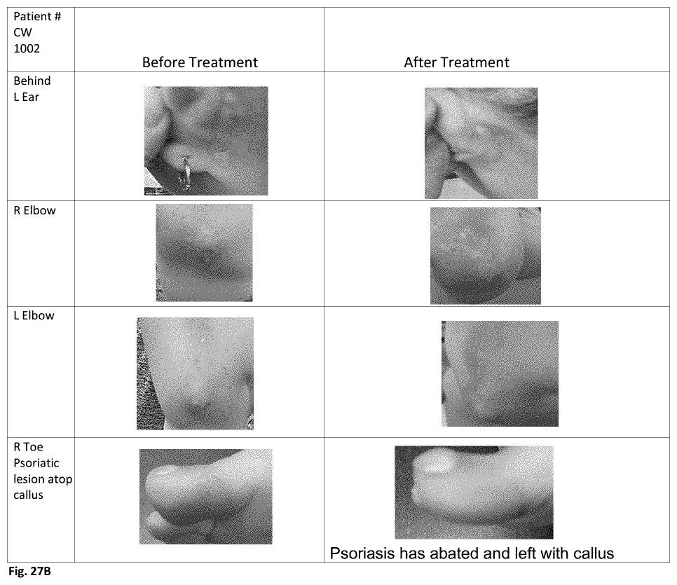

Photographs were taken on select sections of the body showing psoriatic lesions and dryness on both patients on day 0 and at the end of the 12-week study. Results are depicted in FIGS. 26 and 27.

### REFERENCES

Mayo Foundation for Medical Education and Research. (2020, May 2). Psoriasis. Mayo Clinic. Retrieved Sep. 17, 2021, from https://www.mayoclinic.org/diseases-conditions/psoriasis/symptoms-causes/syc-20355840.
Oakley, A. (2009). PASI score. PASI score|DermNet NZ. Retrieved Sep. 17, 2021, from https://dermnetnz.org/topics/pasi-score.
Mosteller R D. Simplified calculation of body-surface area. N Engl J Med. 1987 Oct. 22; 317(17):1098. doi: 10.1056/NE-JM198710223171717. PMID: 3657876.
Finlay, A. Y., & Khan, G. (1994). Dermatology Life Quality Index (DLQI)—a simple practical measure for routine clinical use. Clinical and experimental dermatology, 19(3), 210-216.
Menabde, G., Janelidze, M., & Marks, L. (2017). Use of the universal pain assessment tool for evaluating pain associated with TMD in youngsters with an intellectual disability. Medicina oral, patologia oral y cirugia bucal, 22(1), e88.
Reich, A., Heisig, M., Phan, N. Q., Taneda, K., Takamori, K., Takeuchi, S., . . . & Szepietowski, J. C. (2012). Visual analogue scale: evaluation of the instrument for the assessment of pruritus. Acta dermato-venereologica, 92(5), 497-501.
Bauer, H. (n.d.). Mexameter® MX 18. Courage+Khazaka Electronics. Retrieved Sep. 20, 2021, from https://www.couragekhazaka.de/de/wissenschaftliche-produkte/alle/sondensysteme/16-wissenschaftliche-produkte/alle-produkte/92-mexameter-d.
Bauer, H. (n.d.). Corneometer® CM 825. Courage+Khazaka Electronics. Retrieved Sep. 20, 2021, from https://www.courage-khazaka.de/de/wissenschaftliche-produkte/alle/sondensysteme/16-wissenschaftliche-produkte/alle-produkte/72-corneometer-d.
Bauer, H. (n.d.). Visioscan® VC 20plus. Courage+Khazaka Electronics. Retrieved Sep. 20, 2021, from https://www.courage-khazaka.de/de/wissenschaftliche-produkte/alle/16-wissenschaftliche-produkte/alle-produkte/112-visioscan-d.

### EXAMPLES

### Example 1

One composition of the present disclosure is an anhydrous topical composition (AH) having the ingredients listed below:

| Ingredient (International Nomenclature | Percent in | Percent |
|---|---|---|
| for Cosmetic Ingredients) | Formulation % | Range % |
| MCT Oil Fractioned Coconut Oil | 71.40 | 0.00-95.00% |
| Oleic Acid | 20.00 | 0.00-95.00% |
| Rosemary (Salvia Rosmarinus) | 0.20 | 0.05-1.00% |
| Leaf Extract (and) Ascorbyl | | |
| Palmitate (and) Tocopherol | | |
| Tocopheryl Linoleate | 0.50 | 0.10-2.00% |
| Ascorbyl PM | 0.10 | 0.10-20.00% |
| Ethyl Linoleate | 1.00 | 0.01-5.00% |
| Nigella Sativa (Black Cumin) | 1.00 | 0.01-5.00% |
| Seed Oil | | |
| Cholecalciferol (Vitamin D3) | 0.10 | 0.01-5.00% |
| Bisabolol | 0.20 | 0.01-5.00% |
| Bakuchiol | 1.00 | 0.01-5.00% |
| Full Spectrum Hemp Extract | 2.00 | 0.10-20.00% |
| Cannabigerol (CBG) | 0.10 | 0.01-5.00% |
| Cannabigerol (CBN) | 0.10 | 0.01-5.00% |
| Terepene Blend | 0.50 | 0.10-5.00% |
| Allantoin | 0.10 | 0.01-2.00% |
| Cyanadin | 0.10 | 0.01-1.00% |
| Ceramides | 0.10 | 0.10-10.00% |
| Palmitoylethanolamide | 0.50 | 0.10-20.00% |
| Laurocapram | 1.00 | 0.01-5.00% |

### Example 2

One composition of the present disclosure includes over the counter ingredients (OTC) and has the ingredients listed below:

| Ingredient (International Nomenclature | Percent in | Percent |
|---|---|---|
| for Cosmetic Ingredients) | Formulation % | Range % |
| Water | 67.22 | 0.00-95.00 |
| Allantoin | 0.20 | 0.01-1.00 |
| Xanthan Gum | 1.25 | 0.10-5.00 |
| Glycerin | 6.00 | 0.10-30.00 |
| Glyceryl Stearate (and) PEG-100 | 5.00 | 1.00-20.00 |
| Stearate | | |
| Cetyl Alcohol | 3.00 | 1.00-2.00 |
| Pentaerythrityl Tetracaprylate/ | 3.00 | 0.10-10.00 |
| Tetracaprate | | |
| Dimethicone | 2.00 | 0.10-10.01 |
| MCT Oil Fractioned Coconut Oil | 3.00 | 0.10-30.00 |
| Prunus Amygdalus Dulcis (Sweet | 3.00 | 0.01-10.00 |
| Almond) Oil | | |
| Full Spectrum Hemp Extract | 1.41 | 0.01-10.00 |
| CBD Isolate | 0.67 | 0.01-10.00 |
| Ceramide NP; Ceramide AP; Ceramide | 0.20 | 0.01-10.00 |
| EOP; Phytosphingosine; Cholesterol; | | |
| Sodium Lauroyl Lactylate; Carbomer; | | |
| Xanthan Gum | | |
| Salicylic Acid (and) Dextrin (and) | 5.00 | 0.01-20.00 |
| Polydextrose (and) Amylopectin | | |
| (and) Niacinamide | | |
| Collodial Oatmeal | 0.50 | 0.01-10.00 |
| Ascorbyl Palmitate | 0.20 | 0.01-1.00 |
| Tocopheryl Linoleate | 0.50 | 0.01-1.00 |
| Ethyl Linoleate | 1.00 | 0.10-10.00 |
| Bisabolol | 0.20 | 0.01-1.00 |
| Cholecalciferol | 0.10 | 0.01-1.00 |
| Preservative | 1.00 | 0.01-10.00 |

### Example 3

One composition of the present disclosure is referred to as a relief lotion (RL) and has the ingredients listed below:

| Ingredient (International Nomenclature | Percent in | Percent |
|---|---|---|
| for Cosmetic Ingredients) | Formulation % | Range % |
| Water | 66.10 | 0.00-95.00 |
| Allantoin | 0.20 | 0.01-1.00 |
| Xanthan Gum | 1.25 | 0.10-5.00 |
| Glycerin | 6.00 | 0.10-30.00 |
| Glyceryl Stearate (and) PEG-100 | 5.00 | 1.00-20.00 |
| Stearate | | |
| Cetyl Alcohol | 3.00 | 1.00-2.00 |
| Pentaerythrityl Tetracaprylate/ | 3.00 | 0.10-10.00 |
| Tetracaprate | | |
| Dimethicone | 2.00 | 0.10-10.01 |
| MCT Oil Fractioned Coconut Oil | 3.00 | 0.10-30.00 |
| Prunus Amygdalus Dulcis (Sweet | 3.00 | 0.01-10.00 |
| Almond) Oil | | |
| Full Spectrum Hemp Extract | 1.17 | 0.01-10.00 |
| CBD Isolate | 0.67 | 0.01-10.00 |
| Ceramide NP; Ceramide AP; Ceramide | 0.20 | 0.01-10.00 |
| EOP; Phytosphingosine; Cholesterol; | | |
| Sodium Lauroyl Lactylate; Carbomer; | | |
| Xanthan Gum | | |
| Salicylic Acid (and) Dextrin (and) | 2.00 | 0.01-4.99 |
| Polydextrose (and) Amylopectin | | |
| (and) Niacinamide | | |
| Collodial Oatmeal | 0.50 | 0.01-1.00 |
| Ascorbyl Palmitate | 0.20 | 0.01-1.00 |
| Tocopheryl Linoleate | 0.50 | 0.10-10.00 |
| Ethyl Linoleate | 1.00 | 0.01-1.00 |
| Bisabolol | 0.20 | 0.01-1.00 |
| Cholecalciferol | 0.10 | 0.01-1.00 |
| Preservative | 1.00 | 0.01-10.00 |

### Example 4

One composition of the present disclosure is a psoriasis tincture formulation (TF) and has the ingredients listed below:

| Ingredient (International Nomenclature | Percent in | Percent |
|---|---|---|
| for Cosmetic Ingredients) | Formulation % | Range |
| MCT Oil Fractioned Coconut Oil | 95.00 | 0.00-95.00% |
| Full Spectrum Hemp Extract | 5.00 | 0.10-20.0% |

It will be appreciated by those skilled in the art that while the composition for treatment of psoriasis has been described in detail herein, the invention is not necessarily so limited and other examples, embodiments, uses, modifications, and departures from the embodiments, examples, uses, and modifications may be made without departing from the process and all such embodiments are intended to be within the scope and spirit of the appended claims.

What is claimed is:

1. A composition for the treatment of psoriasis comprising:
at least one CB2 receptor binding agent;
at least one immunosuppressive agent;
at least one oil; and,
at least one healing agent or at least one anti-inflammatory agent.

2. The composition of claim 1 further comprising at least one antioxidant.

3. The composition of claim 2 wherein the at least one antioxidant is vitamin C, vitamin E, vitamin C derivatives, vitamin E derivatives, Rosemary (Salvia rosmarinus) Leaf Extract, ascorbyl palmitate, tocopherol, or mixtures thereof.

4. The composition of claim 1 wherein the at least one CB2 receptor binding agent is a hemp-derived cannabinoid isolate, a hemp-derived full-spectrum cannabinoid containing extract; a hemp-derived broad-spectrum cannabinoid containing extract, or mixtures thereof.

5. The composition of claim 1 wherein the at least one immunosuppressive agent is a terpenoid, cyanidin, cholecalciferol, bisabolol, palmitoylethanolamide, cholecalciferol, or mixtures thereof.

6. The composition of claim 1 wherein the at least one oil is coconut oil, Nigella sativa (Black Cumin) Seed Oil, dimethicone, Prunus amygdalus dulcis (Sweet Almond) Oil, Cannabis sativa (Hemp) Seed oil, or mixtures thereof.

7. The composition of claim 1 wherein the at least one healing agent is allantoin, bisabolol, bakuchiol, ceramides, ethyl linoleate, or mixtures thereof.

8. The composition of claim 1 wherein the at least one anti-inflammatory agent is a cannabinoid, a terpenoid, cyanidin, cholecalciferol, or mixtures thereof.

9. A composition for the treatment of psoriasis comprising:
at least one CB2 receptor binding agent;
at least one oil; and,
at least one healing agent or at least one anti-inflammatory agent.

10. The composition of claim 9 wherein the at least one CB2 receptor binding agent is a hemp-derived cannabinoid isolate, a hemp-derived full-spectrum cannabinoid containing extract; a hemp-derived broad-spectrum cannabinoid containing extract, or mixtures thereof.

11. The composition of claim 9 wherein the at least one oil is coconut oil, Nigella sativa (Black Cumin) Seed Oil, dimethicone, Prunus amygdalus dulcis (Sweet Almond) Oil, hemp oil, or mixtures thereof.

12. The composition of claim 9 wherein the at least one healing agent is allantoin, bisabolol, bakuchiol, ceramides, ethyl linoleate, or mixtures thereof.

13. The composition of claim 9 wherein the at least one anti-inflammatory agent is a cannabinoid, a terpenoid, cyanidin, cholecalciferol, or mixtures thereof.

14. A composition for the treatment of psoriasis comprising:
a topical composition that includes at least one CB2 receptor binding agent; at least one immunosuppressive agent; at least one oil; and, at least one healing agent or at least one anti-inflammatory agent; and,
an ingestible composition that includes a carrier oil MCT Oil Fractioned Coconut Oil, a CB2 receptor binding agent, and Full Spectrum Hemp Extract.

15. The ingestible composition of claim 14 wherein the carrier oil is coconut oil.

16. The composition of claim 14 wherein the at least one CB2 receptor binding agent and the CB2 receptor binding agent is a hemp-derived cannabinoid isolate, a hemp-derived full-spectrum cannabinoid containing extract; a hemp-derived broad-spectrum cannabinoid containing extract, or mixtures thereof.

17. The composition of claim 14 wherein the at least one immunosuppressive agent is a terpenoid, cyanidin, cholecalciferol, bisabolol, palmitoylethanolamide, cholecalciferol, or mixtures thereof.

18. The composition of claim 14 wherein the at least one oil is coconut oil, Nigella sativa (Black Cumin) Seed Oil, dimethicone, Prunus amygdalus dulcis (Sweet Almond) Oil, Cannabis sativa (Hemp) Seed Oil, or mixtures thereof.

19. The composition of claim 14 wherein the at least one healing agent is allantoin, bisabolol, bakuchiol, ceramides, ethyl linoleate, or mixtures thereof.

20. The composition of claim 14 wherein the at least one anti-inflammatory agent is a cannabinoid, a terpenoid, cyanidin, cholecalciferol, or mixtures thereof.
